(19) United States
(12) Patent Application Publication
(10) Pub. No.: US 2005/0251152 A1
(43) Pub. Date: Nov. 10, 2005

(54) Illuminated medicated ink marker

(75) Inventors: Steve A. Herweck, Nashua, NH (US); Jay Zimmerman, Nashua, NH (US)

Correspondence Address: LAHIVE & COCKFIELD, LLP., 28 STATE STREET, BOSTON, MA, 02109, US

(73) Assignee: ATRIUM MEDICAL CORP., Hudson, NH (US)

(21) Appl. No.: 10/839,958

(22) Filed: May 5, 2004

(51) Int. Cl. A61B 17/00

(52) U.S. Cl. 606/116

(57) ABSTRACT

An illuminated medicated ink marker provides a user with the ability to apply and confirm a dosage amount of a drug or agent applied in the form of a liquid, such as an ink, to create the marking, in environments that are otherwise not well lit. The illuminated medicated ink marker includes a holder for controlling the medicated ink marker. A marking portion is coupled with the holder and configured to transfer medicated ink from within the medicated ink marker to a targeted location. An illumination source is coupled with the holder. The illumination source is disposed to illuminate a clinical field including at least a portion of the targeted location.

### FIELD OF THE INVENTION

The present invention relates to a marker, and more specifically to an illuminated marker for use in applying an ink having an active agent. The ink is applied directly to the tissue of a patient, is detectable, and includes at least one medication, drug, and/or therapeutic agent applied to the patient for therapeutic purposes. The illuminated marker illuminates the ink applied.

### BACKGROUND OF THE INVENTION

Application of a therapeutic and/or medical agent to the tissue of a patient, in some instances, occurs through the coating of a medical device with an application of a medical agent for delivering medication to a patient upon usage of the medical device. For example, medical devices, such as balloons or stents, can be coated with one or more agents for controlling restenosis or smooth muscle cell hyperplasia in the human coronary arteries. The balloon or stent can have a drug eluting coating applied to one or more surfaces thereof. With this method, the drug is impregnated or made part of the coating that is applied only to the surface of the medical device structure. Known coating methods provide drug release from a bonded polymeric material or coating that surrounds one or more surfaces of the balloon or stent that generally provide a fixed rate of release of one or more medications.

Alternative to medicated devices, there are often instances where it is desirable to have a drug or agent applied directly to the tissue of a patient. In some instances, there is no need or ability to use a medical device implanted on or in the patient that includes a medicated coating for application to the tissue of the patient. For example, application directly to the skin of a patient can be done without use of a medical device because of easy access to the skin. Alternatively, some applications of medication directly to tissue during surgery may be necessary but without the option of being able to leave an implant within the patient to dispense the medication. If such an implant remains within a patient a subsequent surgery may be required to remove the implant. In other instances it may be desirable to quickly apply medication to specific locations on a patient with specificity. For example, in preparation for a surgical incision, an application of antibiotic, antiseptic, and/or anti-inflammatory agent to the specific incision location could prevent infection and inflammation in and around the surgical incision.

In still another alternative, there are instances where it is desirable to have a drug or agent applied directly to a medial device. For example, the particular drug or agent may not be easily preserved if applied to the medical device at the point of manufacture of the device. However, it may be desirous to have the drug or agent coating on at least a portion of the medical device. As such, the drug or agent can be applied directly to the medical device by the user just prior to application or implantation of the medical device.

An additional consideration is that many drugs or other therapeutic agents that are applied to the tissue of a patient, or to a medical device, are either undetectable or are otherwise not differentiable after application to the tissue. Application of a clear drug or agent can be easily missed upon subsequent inspection. Furthermore, most medications or agents are either clear or white in color, thus differentiating one medication or agent from another is nearly impossible after application to a medical device or tissue. The best way a user of a medical device can ensure that a drug or agent coated on the medical device is the desired drug or agent is if the user applies the drug or agent directly onto the medical device, or tissue, during the surgical procedure from a labeled dispenser of the drug or agent.

Application of the drug or agent directly onto the topical or internal tissue of a patient, or directly to the surface of a medical device, can be carried out using a number of different tools. For example, the drug or agent can be sprayed onto the surface, or painted onto the surface using an applicator designed for dispensing such therapeutic agent. The medical device can also be dipped into a liquid containing the drug or agent, or otherwise applied. A more specific example of such an implementation involves a user dipping a brush, applicator, or other tool into a reservoir of the drug or agent and then using the applicator to apply the drug or agent to the surface of the tissue or medical device. One difficulty with such methods of application in some instances is that it is difficult to visualize internal areas of the body or internal portions of a medical device. As such, the user has difficulty seeing where the drug or agent is being applied. Accordingly, a user may have difficulty in determining how much drug or agent is being applied, and whether it is being applied in the correct location.

### SUMMARY OF THE INVENTION

It is therefore desirable to provide an efficient and accurate device and method for illuminating and applying a medicated ink marking having therapeutic or diagnostic properties directly onto the tissue of a patient or the surface of a medical device. The present invention provides solutions that address this need, in addition to others, as described.

In accordance with one embodiment of the present invention, an illuminated medicated ink marker includes a holder for controlling the medicated ink marker. A marking portion is coupled with the holder and configured to transfer medicated ink from within the medicated ink marker to a targeted location. An illumination source is coupled with the holder. The illumination source is disposed to illuminate a clinical field including at least a portion of the targeted location.

In accordance with aspects of the present invention, the illumination source includes a light emitting source of at least one of a laser, a halogen light, a xenon light, a light emitting diode, a solid fueled light, a liquid fueled light, and a gas fueled light. A controller can be provided for controlling the amount of light emitted from the illumination source. A pressure sensitive switch can control the amount of light emitted from the illumination source.

In accordance with further aspects of the present invention, the illumination source can provide illumination in at least one of the visible and non-visible light spectrums. An amount of light emitted by the illumination source is controllable and variable. The illumination source is removably coupled with the holder. A color of light emitted by the illumination source can be varied by the user. The illumination provided by the illumination source can activate one or more agents within the medicated ink.

In accordance with further aspects of the present invention, a timer can be included for controlling a length of time illumination is provided by the illumination source. The illumination source can include a housing supporting a light emitting source.

In accordance with one embodiment of the present invention, an illumination source coupled with a medicated ink marker includes a housing. A light emitting source is disposed relative to the housing. A controller is provided for controlling a level of light emitted from the light emitting source.

### BRIEF DESCRIPTION OF THE DRAWINGS

The invention will be more fully understood from the following detailed description taken in conjunction with the accompanying drawings, in which:

FIGS. 1A and 1B are diagrammatic illustrations of a marking as applied to a tissue location on a patient, in accordance with aspects of the present invention;

FIGS. 1C and 1D are diagrammatic illustrations of a marking as applied to a tissue location on a patient subsequent to application of a preparatory substance or coating, in accordance with aspects of the present invention;

FIGS. 2A, 2B, and 2C are diagrammatic illustrations of markings applied in different configurations or patterns, in accordance with aspects of the present invention;

FIG. 3 is a diagrammatic illustration of a marking applied around a target area for a surgical incision, in accordance with aspects of the present invention;

FIG. 4 is a diagrammatic illustration of a marking applied around pre-existing wound, in accordance with aspects of the present invention;

FIG. 5 is a diagrammatic illustration of a marking applied as a stamp or decal, in accordance with aspects of the present invention;

FIGS. 6A, 6B, and 6C are diagrammatic illustrations if ink markings applied in different colors, in accordance with aspects of the present invention;

FIG. 7 is a perspective illustration of an illuminated medicated ink marker in accordance with one embodiment of the present invention; and

FIG. 8 is a perspective illustration of an illuminated medicated ink marker in accordance with another embodiment of the present invention.

### DETAILED DESCRIPTION

An illustrative embodiment of the present invention generally relates to improving the conditions under which medications, drugs, therapeutic and/or other agents are applied directly to the tissue of a patient, or to the surface of a medical device in the form of a marking. The present invention provides a clinical user with the ability to apply and confirm a dosage amount of a drug or agent applied in the form of a liquid, such as an ink, to create the marking, in environments that are otherwise not well lit. By use of an illuminating application device, the user can actually apply and control the amount of ink, and thus agent, marked on to the patient or medical device, because the targeted location is better illuminated. The applicator includes an illumination source to light up the targeted location for application of the drug or agent.

The term “markings” as utilized herein is intended to relate to the result of the application of a substance containing a medication, drug, therapeutic agent, adhesive or bonding agent, and/or other agent. The substance can include a form of liquid, ink, or the like, that can be detected by a user with and/or without aid of a device after application. The resulting marking has at least some form of therapeutic or diagnostic benefit to a patient.

The terms “medication” or “medicated” as utilized herein are intended to relate to a substance or use of a substance containing or embodying a drug, agent, therapeutic agent, adhesive or bonding agent, and/or other agent having medicinal or therapeutic benefits.

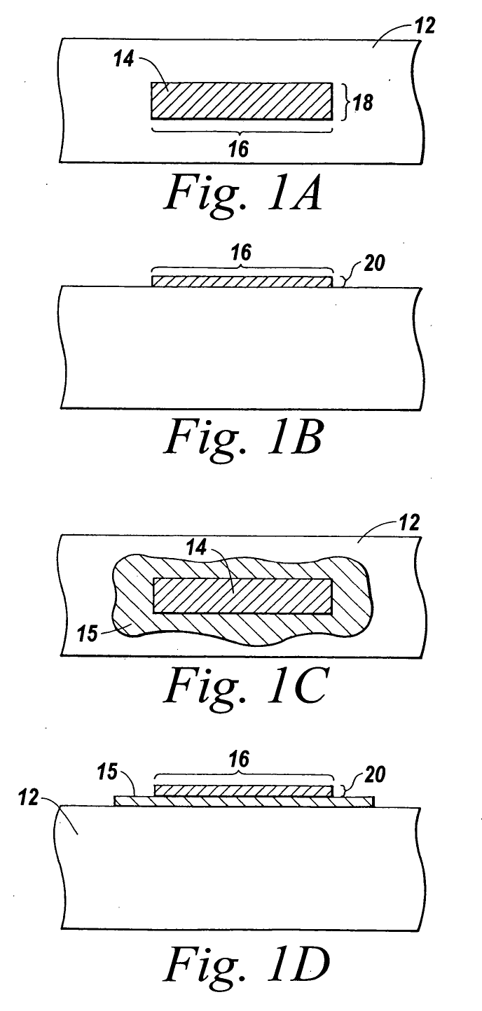

FIGS. 1A through 8, wherein like parts are designated by like reference numerals throughout, illustrate example embodiments of an illuminated medicated ink marker, according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

The teachings of the present invention are applicable both to temporary and permanent markings. A temporarily-placed marking is defined as being a marking that can be removed or will degrade, dissolve, or otherwise dissipate at the conclusion of the therapeutic or diagnostic purpose. A permanently-placed marking, in contrast, stays within the body, or on the surface to which it is applied, for an extended period of time, or in perpetuity.

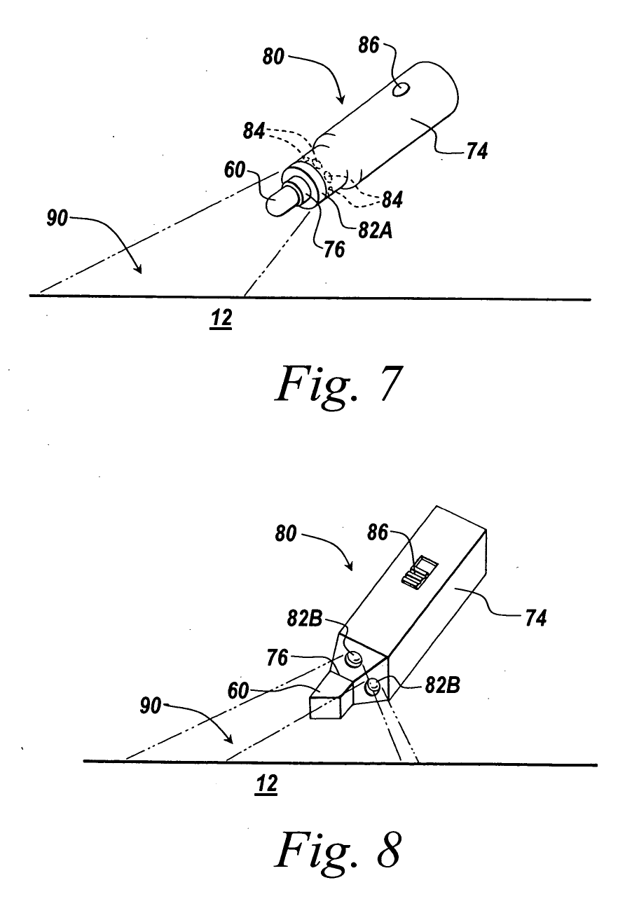

Prior to discussing the illuminated medicated ink marker of the present invention, several examples are offered of different types of markings that can be formed by use of an illuminated medicated ink marker 80 (see FIG. 7).

FIGS. 1A and 1B illustrate examples wherein a marking is applied to a patient or medical device. FIGS. 1A and 1B show a marking 14 that has been applied to a surface 12, such as tissue of a patient, or portion of a medical device. The marking 14 is made by applying an ink that includes an ink carrier component, an agent component, and optionally an adhesive or bonding agent for extended or permanent ink adhesion to the surface 12. Medication saturation, loading, and dimensions of the marking 14 control the dosage of the agent that is delivered to the patient, and ultimately a fixed amount of medication is provided in the illuminated medicated ink marker 80, that provides an upper limit of medication that can be applied. The marking 14 can be made visible, or alternately detectable, by accessory device means, so that the user can confirm the application and the appropriate dosage applied to the surface 12. The marking 14 may be visible, for example, to the naked eye, or under illumination by selected types of light. The dosage of available medication or other agent can also be visibly identified by color or by combination with the dimensions and/or light refraction of the marking 14.

The marking 14 can be applied to the surface 12 in various shapes and forms. FIGS. 1A and 1B show examples where the marking 14 is applied to the surface 12. The marking 14 results from an application that includes an agent component. In one embodiment, the amount of agent in the marking 14 corresponds to the dimensional volume of the marking 14. The dimensional volume of marking applied in FIGS. 1A and 1B is equal to the product of length 16, width 18, and height 20 of the marking 14. The amount of agent on the surface 12 may thus be controlled by varying the dimensions of the marking 14. For example, the amount may be varied by varying the length 16 of the marking 14, the width 18 of the marking 14, or the height 20 (i.e., thickness) of the marking 14. The marking 14 can further be printed in a geometric shape, geometric code, universal bar code, or other format for identification and detection of the agent applied onto the surface 12. As shown in FIGS. 1C and 1D, the amount of the marking 14 deposited can further be increased by altering the surface 12 chemically or otherwise, to alter the ability of the marking to adhere to the surface 12. For example, the surface 12 can have a preparatory layer or coating 15 of a substance that improves absorption of the agent in the marking 14 by the surface 12. The layer or coating 15 can have a number of other results, such as enabling the marking 14 to better adhere to the surface 12, or to react with the marking 14 upon application of the marking 14 to the surface 12. The layer or coating 15 can be applied immediately before application of the marking 14, or can be applied at periods of time substantially before application of the marking 14 to have a more extensive effect on the surface 12.

The surface area of the marking 14 can also affect the rate of delivery of the agent to the patient. In general, a larger surface area results in a higher rate of delivery of the agent than a smaller surface area (given a same concentration of agent). Further, an irregular surface topography, including pores, may either increase or decrease the amount of marking applied to the surface 12. Hence, a clinical user may wish to consider both the volume and surface area when marking the surface 12.

More specifically, the markings 14 can have different lengths and thicknesses chosen for delivery of the appropriate dosages of the medical agents. In other words, given a uniform number of application layers, increased lengths of markings 14 result in increased dosages of the agents. Therefore, upon quick visual inspection, a user can determine and/or confirm the dosage amount provided. If the thickness is varied, the same length of marking 14 can also result in different dosages. Again, the upper limit of the dosage is mandated by the total amount of drug or agent contained within the illuminated medicated ink marker 80, because there is no reservoir or other source that can be re-visited by the user for additional medication.

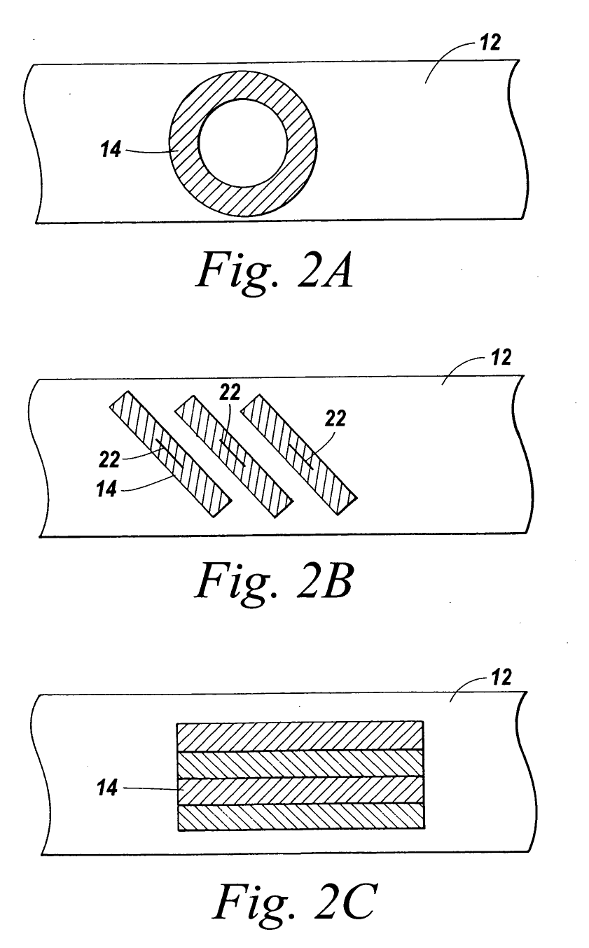

As previously mentioned, the marking 14 can be applied to the surface 12 in various shapes and forms. FIGS. 2A, 2B, and 2C show examples where the marking 14 is applied to the surface 12. The marking 14, as applied by a clinical user, can have an essentially infinite number of patterns or designs. FIG. 2A shows the marking 14 in a generally circular shape. The circle can be hollow, as shown, or solid. The circle can be placed on the surface 12 in a manner that surrounds a wound or other identifiable area on the surface 12 requiring treatment. The marking can also be placed on top of such an area.

FIG. 2B shows an additional example of the marking 14 in a pattern of angled lines. The lines are disposed over a medical fastening device 22, such as stitches or a staple. The illustration represents the use of the marking 14 as, for example, an anti-inflammatory, anti-microbial, or anti-infective agent place over the medical fastening device 22 to prevent infection. Either before, or after, insertion of the medical fastening device 22, the markings 14 are placed on the surface 12 in the approximate location of the medical fastening device 22. The agents contained within the marking 14 can be varied for the particular application. Those agents listed relative to FIG. 2B are merely illustrative of example agents or medications.

FIG. 2C shows an example of the marking 14 formed of a series of parallel lines. The parallel lines can be formed of the same ink with the same agent or agents. As shown, the lines are formed of at least two different inks and agents. This illustration shows how multiple inks and agents can form the marking 14 as applied to the surface 12. With different inks, and more particularly different agents, multiple symptoms or maladies can be treated simultaneously. The different inks and agents can form the markings 14 in whatever combination the clinical user desires, to achieve whatever therapeutic effect attributable to the particular agents being applied in the markings 14.

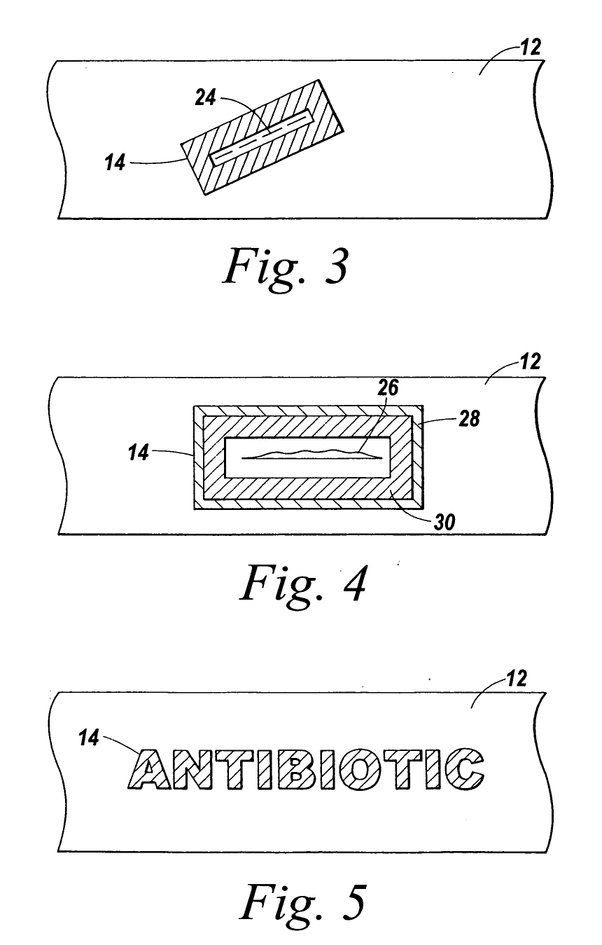

FIG. 3 shows the marking 14 in the general shape of a hollow rectangle. Inside the hollow rectangle shape of the marking 14, a dotted line 24 indicates the location of a future surgical incision. The marking 14 in such an instance can contain a therapeutic agent, such as a sterilization, anti-inflammatory, anti-microbial, and/or anti-infective agent, or some other agent as understood by one of ordinary skill in the art. The marking 14 can both serve to reduce the likelihood of infection of the pending incision, and also serve to help the surgeon visibly identify the location for making the incision. If desired, the marking 14 can be made in such a way as to indicate the desired direction, depth, or other characteristics of the pending incision.

FIG. 4 shows the marking 14 again in the general shape of a hollow rectangle. However, in the example embodiment shown the marking 14 surrounds an existing incision or wound 26 on the surface 12 of the patient. If the marking 14 is not present prior to the incision or wound 26 as described in FIG. 3, the marking 14 can be made after the existence of the wound 26 for therapeutic purposes. The marking 14 of FIG. 4 additionally demonstrates an example embodiment wherein the marking 14 is made of two different markings containing two different agents. A first marking 28 and a second marking 30 surround the incision or wound 26. As depicted, the first marking 28 and second marking 30 can be applied in two different arrangements. The first marking 28 can serve as a border that surrounds the second marking 30. In this instance, the agent(s) in the second marking 30 are closer to the incision or wound 26, and thus have a more immediate effect, while the agent(s) in the first marking 28 are more removed from the incision or wound 26, thus having a secondary or delayed effect. Alternatively, the first marking 28 can be applied to the surface 12 and then the second marking 30 can be applied directly on top of the first marking 28 to form a layered effect. In such an instance, the agent(s) in the first marking 28 are closest to the surface 12 and the incision or wound 26, thus having a primary effect on the tissue. The agent(s) in the second marking 30 must either wait for the first marking 14 to be absorbed by the surface 12, or pass through the first marking 28 to reach the surface 12. Thus, the agent(s) in the second marking 30 have a secondary effect on the surface 12.

One of ordinary skill in the art will appreciate that there can be any number of layers as shown in FIG. 4 having the same dimensions or different dimensions as applied to the surface 12. The different layers can contain the same or different agents. For example, to increase the dosage of a particular agent in a specified location on the surface 12, multiple layers of markings 14 can be made over the specified location. Each layer is an added dosage amount. Alternatively, different agents can exist in each layer. Thus, for example, an agent that improves tissue absorption can form the first layer or first marking 28, and the therapeutic agent can exist in the second layer or second marking 30 applied on top of the first marking 28. Alternatively, two or more components of a therapeutic agent can be applied in separate markings. For example, the first marking 28 can include a first component of a therapeutic agent, while the second marking 30 can include a second component of the therapeutic agent. Once the second marking 30 is applied over the first marking 28, each of the components combines to form the therapeutic agent formed on the surface 12 for the desired therapeutic effect. In addition, the application of the layers can be staggered. For example, the first marking 28 can be applied including a therapeutic agent that has a therapeutic effect on the surface 12. After a selected period, the second marking 30 is then applied, resulting in an additional therapeutic effect. Such a process can continue as desired with additional layers of markings.

FIG. 5 shows another example embodiment of the marking 14. In this instance, the marking 14 is in a predetermined form, symbol, or word. As shown, the marking 14 is in the form of the word “antibiotic”, which would indicate that the marking 14 includes at least one antibiotic agent. The marking 14 in this instance can be applied by the user writing the desired word using the illuminated medicated ink marker 80. One of ordinary skill in the art will appreciate that the form, symbol, word, and the like, can take many different forms and can convey information as desired.

The present invention enables a physician to apply the marking 14 at a desired location on the surface 12 of a patient or medical device. For example, a user can apply antibiotic, analgesic, or anti-inflammatory medicated ink marks on a specific location where the medicated ink marks will provide the most therapeutic benefit. Further, a user can also apply a medicated ink mark to the specific desired location of dialysis needles, dialysis catheters, orthopedic implant or traction pins, laparoscopic devices, or spinal tap needles with detectable confirmation and/or visual confirmation prior to or during medical device usage.

A combination or mixture of a non-medicated ink or other substance with the ink containing the agent to form a blended ink is another method for controlling the rate of delivery of the agent to the patient. With the addition of the non-medicated ink or substance, the amount and rate of activation and/or release of the agent can be made different for different agents and/or different anatomical locations. A second non-medicated ink can further be applied as the second marking 30 to modulate the activation and/or release of the agent from the first marking 28. In addition, the surface 12 can be pre-treated with a medicated or non-medicated substance to affect absorption by the tissue.

Numerous modifications to marking shape, including pattern and orientation, will be apparent to those skilled in the art in view of the foregoing description. Accordingly, this description is to be construed merely as illustrative of the inventive concept herein. The description and illustrations should not be construed as limiting the invention.

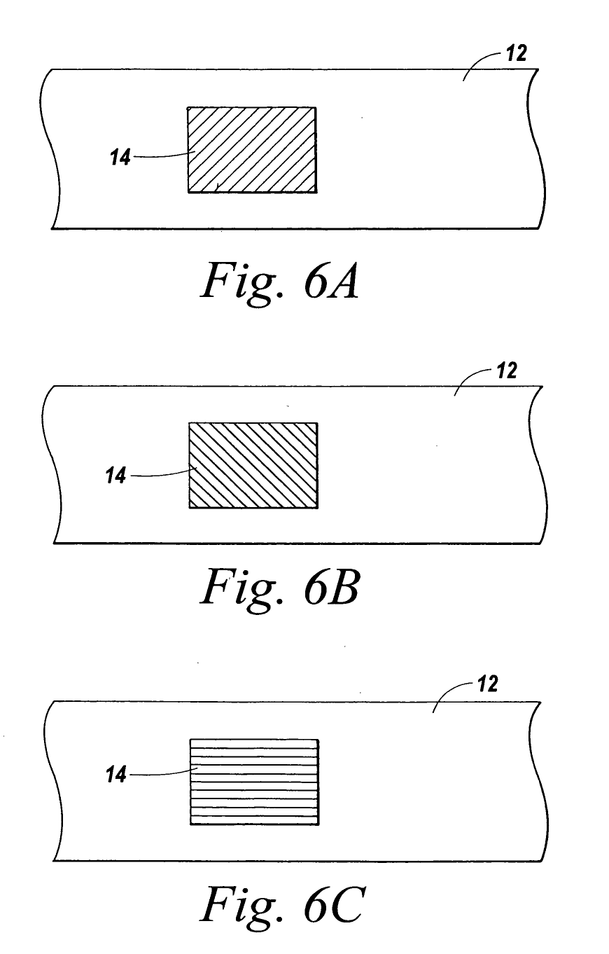

FIGS. 6A, 6B, and 6C illustrate three different embodiments of the marking 14, in the form of three different colors. FIG. 6A shows the marking 14 having a first color. FIG. 6B shows the marking having a second color. FIG. 6C shows the marking having a third color. The marking 14 is shown in the same generally rectangular shape, however, the shape of the marking 14 can vary regardless of the color.

Those skilled in the art will appreciate that a number of different bio-erodable, soluble, or permanent marker inks may be used to create the marking 14. In general, inks are formulated using a pigment to impart color, a resin binder to form the finished ink and carry the pigment, drug exuding medication, or chemical and/or solvent required to enable the binder-pigment mixture to be adhered to the tissue. Suitable pigments include but are not limited to those approved by the USFDA for medical use as listed in Title 21, Sections 73 and 74 of the Code of Federal Regulations (CFR). The following are directly applicable to tissue:

| | |
|---|---|
| Ultramarine blue | FD&C Blue |
| Iron oxide | FD&C Green |
| Titanium oxide | FD&C Red |
| Chromium-cobalt-aluminum oxide | FD&C Yellow |
| Ferric ammonium citrate | D&C Orange |
| Chromium oxide green | D&C Brown |
| Logwood extract | D&C Violet |
| Phthalocyanine green | |

In addition, those of ordinary skill in the art will appreciate that the colors can provide an indication of agent brand name, or an indication of type of agent, associated with the marking 14, as a confirmation of information conveyed by a label 62 of the illuminated medicated ink marker 80. For example, if a particular drug has a unique color associated with its identification or trademark, the same color can be replicated in the ink of the marking 14, such that the marking 14 is easily identified as containing that particular drug or agent. Alternatively, the color of the marking 14 can provide an indication of a type of agent found in the marking 14 applied. The use of different colors allows a physician, or other clinical user, to visibly identify the class of medication applied to the surface 12. The different color schemes for different classification types of medication provide the user with the ability to check and confirm prior to incision or other action, which medication or therapeutic application is incorporated into the ink applied to the surface 12. The specific color scheme utilized can be standardized by, for example, a national standardizing entity. The color scheme can include solid colors, as shown in FIGS. 6A through 6C, or can include simple patterns of alternating or otherwise differing colors. In addition, the color can be one that is only visible when certain light wavelengths are directed toward the color, such as UV light directed toward an iridescent color. One of ordinary skill will appreciate the virtually infinite variability of colors, hue, fluorescence, and simple color patterns that can be used to identify particular classes or types of drugs. The colors can identify specific brand names of drugs, or any other desired clinically related attribute, as well.

As previously indicated, medical agents may be added directly to ink formulations to provide the marking 14 with medical properties. Additives and drug carrying nano-particles or microspheres containing medical agents may also be included in the ink formulation to achieve specific rates of medication permeation to local tissue. For example, fast soluble and slow soluble nano-particles or microspheres, organic solvents, and surfactants may be used to achieve a desired ink viscosity to apply the ink onto the surface 12. The solvent and surfactant are optionally removed in a subsequent process step. Other additives can include plasticizers, bio-erodable components, dye components, adhesives, bonding agents, medication stabilizers, coated and non-coated medical agent nano-particles, or microspheres, designed to improve the ink's flexibility, flow, pigment stability, shelf-life stability, and rate of surface activation and/or release into tissue or body fluid. Inks can also be formulated containing liposomes, with medication enclosed in liposomes, or phospholipid coatings. These inks can be triggered to release active compounds using an internal or external stimulus, such as ultrasound, radiation, magnetic field, or temperature, and can also be cured with application of light, such as UV light.

The following examples illustrate exemplary embodiments of the present invention. A first example involves the use of the present invention in surgery. In particular, a user can make use of a visually detectable marking 14 in orthopedic surgery. In such a surgical procedure, it is often the case that there is a significant amount of blood or other fluids in the vicinity of the procedure. The user can apply the marking 14, and because it can be made with an ink that is highly visually detectable, the user can see where the therapeutic has been applied.

Another application involves laparoscopic surgery, whereby internal tissue visualization and surgical intervention is done solely by video camera and port sealed instrumentation. A laparoscope is placed through a small incision or opening in the patient. The video image is then transmitted back to a video monitor so the surgeon can see where the laparoscope is within the patient. Use of a visually detectable medicated or therapeutic ink by a suitable laparoscopic surgical instrument to form a marking 14 on the surface 12 internal to the patient facilitates application control and confirmation of therapeutic delivery to the targeted location. The illuminated medicated ink marker 80 of the present invention has particular application in such instances because the illumination from the laparoscope may not be sufficient, due to the medicated ink marker 80 blocking the light from the laparoscope. In such instances, the additional illumination provided at the specific point of medication application by the illuminated medicated ink marker 80, as later discussed, can provide the needed light.

Still another application of the present invention involves the use of radiopaque or otherwise machine detectable ink. In such an instance, the stability or migration of the therapeutic agent applied to a specific targeted location can be confirmed non-invasively by ultrasound, x-ray, MRI, CAT, PET, and the like. For example, the ink can be applied to a specific location during a surgical procedure. Hours or days later, the stability of the ink, or the migration of the ink, can be verified by remote monitoring because of the machine detectable qualities of the ink.

Those skilled in the art will appreciate that a number of different medical agents may be used in the marking 14. For example, anesthetic, anti-infective, lipid lowering, absorption enhancing, anti-oxidant, anti-platelet, cytostatic or cytotoxic medications can be used. In addition, medical agents that promote hollow fluid organ vaso dilation, vaso constriction, occlusion, or thrombosis can be used. The medical agents may include drugs that promote anti-thrombotic activity or can be a clot lysing agent known as a thrombolytic. The medical agents can be kinases or enzymes. The medical agents can be those that promote anti-inflammatory activity or those that promote or stimulate new bone growth. The medical agents can further include agents that promote new cell growth and/or tissue regeneration. The table below (Table #1) summarizes some examples of suitable therapeutic medical agents listed by class.

TABLE #1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Antiinflammatory Agents | Glucocorticoids, NSAIDS, ibuprofen, acetaminophen, |
| | hydrocortizone acetate, hydrocortizone sodium phosphate |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | microphenonol acid, rapamycin, everolimus, tacrolimus, |
| | paclitaxel, estradiol, dexamethasone, methatrexate, |
| | cilastozol, prednisone, cyclosporine, doxorubicin, |
| | ranpirnas, troglitzon, valsarten, pemirolast |
| Tissue growth stimulants | Bone morphogeneic protein, fibroblast growth factor |
| Gasses | Nitric oxide, super oxygenated O2 |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estrogen, nitric oxide |
| delivery | |
| Second messenger | Protein kinase inhibitors |
| targeting | |
| Angiogenic | Angiopoetin, VEGF |
| Anti-Angiogenic | Endostatin |
| Inhibitation of Protein | Halofuginone |
| Synthesis | |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride, Selenium. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donative | NCX 4016 - nitric oxide donative derivative of aspirin, |
| Derivatives | SNAP |
| Gases | Nitric oxide, super oxygenated O₂ compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochlorite |
| Chemotherapeutic Agents | Cyclosporine, doxorubicin, paclitaxel, tacrolimus, |
| | sirolimus, fludarabine, ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyalonic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |
| Antiseptics | Selenium |

In addition to or in conjunction with the above table, the medical agent of the present invention can further include an antimicrobial agent. As utilized herein, the term antimicrobial agent shall include antibiotic, antimicrobial, antibacterial, germicidal agents and the like. There may be a combination of antimicrobial agents. In addition, example antibiotics which may be used in conjunction with the present invention include: aminoglycosides, such as gentamicin, kanamycin, neomycin, paromomycin, streptomycin, or tobramycin; ansamycins, such as rifamycin, or rifampin; cephalosporins, such as cephalexin, cephaloridine, cephalothin, cefazolin, cephapirin, cephradine, or cephaloglycin; chloramphenicols; macrolides, such as erythromycin, tylosin, oleandomycin, or spiramycin; penicillins, such as penicillin G and V, phenethicillin, methicillin, oxacillin, cloxacillin, dicloxacillin, floxacillin, nafcillin, ampicillin, amoxicillin, or carbenicillin; suflonamides; tetracyclines, such as tetracycline, oxytetracycline, chlortetracycline, methacycline, demeclocycline, rolitetracycline, doxycycline, or minocycline; trimethoprim-sulfamethoxazole; polypeptides, such as bacitracin, polymyxins, tyrothricin, or vancomycin; and miscellaneous antibiotics, such as lincomycin, clindamycin, or spectinomycin, in addition to oxytetracycline hydrochloride (OTC).

There are a plurality of germicides which may at least partially form the medical agent of the present invention, including phenols; cresols; resorcinols; substituted phenols; aldehydes; benzoic acid; salicyclic acid; iodine; iodophors, such as betadine; chlorophors, such as hypochlorites; peroxides; such as hydrogen peroxide and zinc peroxide; heavy metals and their salts, such as merbromin, silver nitrate, zinc sulfate; surface-active agents, such as benzalkonium chloride; furan derivatives, such as nitrofurazone; sulfur and thiosulfates; salicylanilides; and carbanilides.

The amount of the antibiotic, bactericidal, or germicide present in an application of a marking varies with the nature of antibiotics or germicides employed and to some extent the method applying the marking as understood by one of ordinary skill in the art.

FIG. 7 is a perspective illustration of the illuminated medicated ink marker 80 in the form of a medicated porous applicator 60 and a holder 74 with an illumination source 82 coupled thereto to form the illuminated medicated ink marker 80. The embodiments illustrated, as well as equivalents as understood by one of ordinary skill in the art, are referred to herein with the general reference of the illuminated medicated ink marker 80. However, the present invention is not limited to the embodiments illustrated, but rather anticipates other shapes and forms of the illuminated medicated ink marker 80 that can perform the stated functions as described herein.

The medicated porous applicator 60 portion of the illustrative embodiment of the illuminated medicated ink marker 80 is formed of a generally porous material, such as a plastic, composite, rubber, rubberized plastic or composite, porous synthetic, and the like. As discussed above, the material of the illuminated medicated ink marker 80 forms a wick that maintains wicking characteristics. By wicking characteristics, what is meant is that although porous, the material forming the illuminated medicated ink marker 80 is configured to create capillary action to draw liquid from one end to the other of the material. When the medicated porous applicator 60 makes contact with the surface, the capillary action initiates, and the fluid contained within the porous material wicks out to the surface 12.

In accordance with one embodiment, the medicated porous applicator 60 portion is saturated with the drug or agent to an extent such that a predetermined dosage amount of the drug or agent is held within the medicated porous applicator 60. As the medicated porous applicator 60 makes contact with the surface 12 a wicking action draws the drug or agent from the medicated porous applicator 60 to the surface 12.

In accordance with one embodiment of the medicated porous applicator 60, the entire dosage of the drug or agent is contained within the porous medicated porous applicator 60. There is no reservoir connected with the porous illuminated medicated ink marker 80 from which the medicated porous applicator 60 can draw any drug or agent. Accordingly, once the illuminated medicated ink marker 80 is utilized on the desired surface 12, the illuminated medicated ink marker 80 is not reused and is disposed of by the user. The illumination source 82 can be decoupled from the medicated porous applicator 60 for later reuse prior to disposal of the marker.

The medicated porous applicator 60 fits within the holder 74, an example embodiment of which is shown in FIG. 7. The holder 74 has a coupling 76 for receiving the medicated porous applicator 60, the specific mechanism of which can vary as understood by one of ordinary skill in the art, and can include adhesive, mechanical fastener, and the like. The holder 74 is a structure that is more easily manipulated by the user when applying the medicated porous applicator 60 against the surface 12. The holder 74 represents any number of different variations of tools or implements for holding the medicated porous applicator 60 to form the illuminated medicated ink marker 80. The different variations must also include some form of illumination, such as the illumination source 82 shown in the figures.

The illuminated medicated ink marker 80, as mentioned, includes the illumination source 82. The illumination source 82 can take many different forms, some of which are illustrated in FIGS. 7 and 8 as illumination source 82a and illumination source 82b. For example, illumination source 82a is a generally transparent ring with a series of bulbs 84 located behind the transparent ring. The bulbs 84 emit light through the transparent ring toward the surface 12 upon which the drug or agent is to be applied to highlight a clinical field 90. As such, the light emits in a generally 360° pattern surrounding the medicated porous applicator 60 and providing complete illumination of the clinical field 90 and the surface 12 in front of the illuminated medicated ink marker 80.

The illumination source 82 of the illuminated medicated ink marker 80 can have a controller 86 that controls the illumination source 82. The controller can have many different configurations. For example the controller 86 can be a pressure sensitive switch, an on-off switch, a push-button switch, an infinitely variable switch, and the like, as would be understood by one of ordinary skill in the art. The controller 86 generally controls whether the illumination source 82 is on or off, and/or the intensity of the illumination source 82. In addition, the controller 86 can include a timer feature, such that the user can initiate illumination using the controller and after a predetermined time period the controller 86 can automatically shut off the illumination, thus indicating that the time period had passed. This can be useful in the application of certain medications that are time dependent, such as UV cured substances.

The illumination source 82 of the illuminated ink marker 80, to further elaborate on the light curable substances, can be utilized, for example, to activate the drug or agent by providing a curing function, or enhance the application, absorbancy or adhesion of the therapeutic agent or drug.

In FIG. 8, there are two separate illumination sources 80b in the illuminated medicated ink marker 80. The two separate illumination sources 80b can be more precisely positioned than the transparent ring of the previous embodiment, such that light can be specifically directed to a more focused clinical field 90, or a more diverse clinical field 90, if desired. In addition, the number of separate illumination sources 80b can vary, as would be understood by one of ordinary skill in the art.

One of ordinary skill in the art will further appreciate that the number and type of light sources can vary. For example, light from a single source can be dispersed to cover a relatively wide area. The specific light pattern can be manipulated by location of the light source and the specific lens or transparent component through which the light passes. The light source itself can be an incandescent bulb, an LED, a halogen bulb, a Xenon bulb, a laser, a solid fueled light, a liquid fueled light, a gas fueled light and the like, such that the specific form of light source is not limited to the embodiments illustrated. Furthermore, the light source can have wavelengths that fall within specific areas of the light spectrum, in both visible and non-visible wavelengths. For example, ultraviolet light (UV light) can be useful in highlighting iridescent inks. In addition, the light source can contain several different bulbs, such that the type of light can be altered or changed by the clinical user while using the illuminated medicated ink marker 80.

The illumination source 82 can further be permanently mounted to the holder 74, or can be removably coupled. For example, the illumination source 82a of FIG. 7 can be held in place with a threaded fitting between the coupling 76 and the body of the holder 74. In FIG. 8, the illumination source 82b can be held in place with a friction fitting, and installed or removed through the back end of the holder 74. In general, the illumination source 82 can be coupled to the body of the holder 74 via mechanical fastener or other removable coupling such that the illumination source 82 can be removed when the medicated porous applicator 60 has been emptied of the drug or agent and is set for disposal, such that the illumination source 82 can be reused, if desired.

The illuminated medicated ink marker 80 containing the ink can be used to apply the marking 14 to the surface 12. The clinical user draws the desired marking 14 directly on the surface 12 with the ink containing one or more therapeutic agents. Different color medicated ink markers 60 can contain different medication classifications or types of medication based on different color schemes. The illuminated medicated ink marker 80 can also be utilized in forming simple color patterns, symbols, or text.

The markings 14 of the present invention enable the distribution of agents to a targeted location on a patient's body. The ink is relatively thin and unobtrusive to the applied surface. The marking 14 can further provide relevant information concerning the agents combined with the ink, as well as other characteristics of the ink and/or the agent, such as drug type, drug brand, drug dosage, dimensions, sizing, placement, orientation, and the like.

The present invention has many different therapeutic uses. More specifically, one clinical use for the marking 14 containing at least one agent is for application onto the surface 12. The surface 12 can include both internal and external sides of a patient's skin, as well as any other tissue within the patient. In some instances, the tissue may only be accessible during a surgical or other medical procedure.

All identifiable and/or detectable drug exuding inks that form the markings 14 can be made as a permanent marking or as a temporary marking, which can be absorbed by the local surface 12. More specifically, the marking 14 can have a relatively short term therapeutic effect, or the marking 14 can have a longer term, more permanent effect. A tattoo, for example, is representative of an ink that is a longer term application. Whereas, an ink that is applied and is absorbed in a matter of minutes or days has a shorter term therapeutic effect. Inks and agents combined with inks can have therapeutic effects ranging between the shorter term and longer term applications.

The present invention, thus, provides an illuminated medicated ink marker that, as a part of the drug or agent dispensing applicator, can illuminate a targeted location for the delivery of a drug or agent to a desired surface, such as tissue or the surface of a medical device. The illumination of the targeted location can be accomplished using one or more bulbs in combination with a transparent component or lens that can disperse and direct the light as desired.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. It is intended that the present invention be limited only to the extent required by the appended claims and the applicable rules of law.

1. An illuminated medicated ink marker, comprising:
a holder for controlling the medicated ink marker;
a marking portion coupled with the holder and configured to transfer medicated ink from within the medicated ink marker to a targeted location; and
an illumination source coupled with the holder;
wherein the illumination source is disposed to illuminate a clinical field including at least a portion of the targeted location.

2. The medicated ink marker of claim 1, wherein the illumination source comprises a light emitting source of at least one of a laser, a halogen light, a xenon light, a light emitting diode, a solid fueled light, a liquid fueled light, and a gas fueled light.

3. The medicated ink marker of claim 1, further comprising a controller for controlling the amount of light emitted from the illumination source.

4. The medicated ink marker of claim 1, further comprising a pressure sensitive switch for controlling the amount of light emitted from the illumination source.

5. The medicated ink marker of claim 1, wherein the illumination source provides illumination in at least one of the visible and non-visible light spectrums.

6. The medicated ink marker of claim 1, wherein an amount of light emitted by the illumination source is controllable and variable.

7. The medicated ink marker of claim 1, wherein the illumination source is removably coupled with the holder.

8. The medicated ink marker of claim 1, wherein a color of light emitted by the illumination source can be altered.

9. The medicated ink marker of claim 1, wherein the illumination provided by the illumination source activates one or more agents within the medicated ink.

10. The medicated ink marker of claim 9, wherein the activation of one or more agents comprises the activation of at least one of curing, enhanced application, enhanced absorbancy, and enhanced adhesion of the medicated ink.

11. The medicated ink marker of claim 1, further comprising a timer for controlling a length of time illumination is provided by the illumination source.

12. The medicated ink marker of claim 1, wherein the illumination source comprises a housing supporting a light emitting source.

13. An illumination source coupled with a medicated ink marker, comprising:
a housing;
a light emitting source disposed relative to the housing; and
a controller for controlling a level of light emitted from the light emitting source.

14. The illumination source of claim 13, wherein the light emitting source comprises at least one of a laser, a halogen light, a xenon light, a light emitting diode, a solid fueled light, a liquid fueled light, and a gas fueled light.

15. The illumination source of claim 13, wherein the controller comprises a pressure sensitive switch for controlling the amount of light emitted from the light emitting source.

16. The illumination source of claim 13, wherein the light emitting source provides light in at least one of the visible and non-visible light spectrums.

17. The illumination source of claim 13, wherein an amount of light emitted by the light emitting source is controllable and variable.

18. The illumination source of claim 13, wherein the illumination source is removably coupled with the medicated ink marker.

19. The illumination source of claim 13, wherein a color of light emitted by the light emitting source can be altered.

20. The illumination source of claim 13, wherein the illumination provided by the light emitting source activates one or more agents within the medicated ink.

21. The illumination source of claim 20, wherein the activation of one or more agents comprises the activation of at least one of curing, enhanced application, enhanced absorbancy, and enhanced adhesion of the medicated ink.

22. The illumination source of claim 13, further comprising a timer for controlling a length of time illumination is provided by the light emitting source.
